(19) United States
(12) Patent Application Publication
(10) Pub. No.: US 2021/0145345 A1
(43) Pub. Date: May 20, 2021

(54) ELECTRODE EXTENSION ASSEMBLY FOR A PORTABLE ECG AND PCG SENSOR DEVICE

(71) Applicant: COALA-LIFE AB, Stockholm (SE)

(72) Inventors: Magnus SAMUELSSON, Stockholm (SE); Philip SIBERG, Stockholm (SE)

(21) Appl. No.: 17/256,322

(22) PCT Filed: Jun. 18, 2019
(86) PCT No.: PCT/SE2019/050581

§ 371 Date: Dec. 28, 2020

(30) Foreign Application Priority Data
Jun. 29, 2018 (SE) 1850808-5

(51) Int. Cl. A61B 5/332; A61B 7/04; A61B 5/0205; A61B 5/274; A61B 5/282; A61B 5/257

(52) U.S. Cl. CPC A61B 5/332; A61B 7/04; A61B 5/0205; A61B 5/6823; A61B 5/282; A61B 5/257; A61B 5/274

(57) ABSTRACT

It is provided an electrode extension assembly comprising: a fastener for releasably fastening the electrode extension assembly to a user; a device holder for releasably holding a portable sensor device configured to capture electrocardiogram signals and audio signals; at least two extenders extending radially outwards from the device holder, wherein each extender comprises: a connector configured to connect to a respective electrode of the portable sensor device; an outer electrode connected at a distal end of the extender, the outer electrode being configured to be applied to a user; a conductor connected on one end to the connector and on the other end to the outer electrode; and an audio signal conveyor for conveying audio signals from the user to an audio sensor of the portable sensor device.

### TECHNICAL FIELD

The invention relates to an electrode extension assembly and corresponding kit for fastening a portable sensor device to the body of a user.

### BACKGROUND

ECG is an established technology where electric signals generated by the body of a patient are measured and analysed. Traditionally, a number of electrodes are placed on the body at various places. A conductive gel is used to provide better conductive contact between the electrodes and the skin. The patient typically lies down for minutes when the ECG is taken. The data detected using the electrodes is recorded and can be analysed by a professional, such as a physician or trained nurse. Once the measurement procedure is done, the conductive gel is wiped off.

While having proved useful, the traditional way of obtaining an ECG is not optimal in all cases. For instance, such an ECG needs to be measured in a clinic and the procedure is messy for the patient.

Lately, portable sensor devices with integral electrodes for obtaining ECG data have been developed. These portable sensor devices allow users to capture ECG data at will and also without the use of conductive gel. This gives the user greater control over when to capture ECG data and also in a much more convenient and less messy way. Such portable sensor devices can also be configured to measure phonocardiogram (PCG) data, i.e. sound data of the heart.

However, a user still needs to actively decide when to capture ECG and PCG data. It would be of great value if ECG and PCG measurements could be captured easier, without requiring the user to actively start the measurement.

### SUMMARY

It is an object to simplify capturing of ECG and PCG measurement using a portable sensor device.

According to a first aspect, it is provided an electrode extension assembly comprising: a fastener for releasably fastening the electrode extension assembly to a user; a device holder for releasably holding a portable sensor device configured to capture electrocardiogram signals and audio signals; at least two extenders extending radially outwards from the device holder, wherein each extender comprises: a connector configured to connect to a respective electrode of the portable sensor device, when located in the device holder; an outer electrode connected at a distal end of the extender, the outer electrode being configured to be applied to a user, when the electrode extension assembly is fastened to a user; a conductor connected on one end to the connector and on the other end to the outer electrode; and an audio signal conveyor for conveying audio signals, when the electrode assembly is fastened to a user and a portable sensor device is located in the device holder, from the user to an audio sensor of the portable sensor device.

The fastener may comprise an adhesive for fastening the electrode extension assembly to a user.

The electrode extension assembly may comprise three extenders.

The audio signal conveyor may be a through hole provided, when the electrode assembly is fastened to a user and a portable sensor device is located in the device holder, between the user and the audio sensor of the portable sensor device.

The device holder may comprise a snap fastener.

The electrode extension assembly may be disposable.

According to a second aspect, it is provided a sensor kit comprising the electrode extension assembly according to any one of the preceding claims and a portable sensor device to be located in the device holder of the electrode extension assembly.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

The invention is now described, by way of example, with reference to the accompanying drawings, in which:

FIGS. 1A-B are schematic diagrams illustrating an environment in which embodiments presented herein can be applied;

FIGS. 2A-2B are schematic diagrams of views illustrating a physical representation of the portable sensor device of FIG. 1 according to one embodiment; and

FIGS. 3A-B are schematic diagrams illustrating the electrode extension assembly of FIG. 1 according to one embodiment.

### DETAILED DESCRIPTION

The invention will now be described more fully hereinafter with reference to the accompanying drawings, in which certain embodiments of the invention are shown. This invention may, however, be embodied in many different forms and should not be construed as limited to the embodiments set forth herein; rather, these embodiments are provided by way of example so that this disclosure will be thorough and complete, and will fully convey the scope of the invention to those skilled in the art. Like numbers refer to like elements throughout the description.

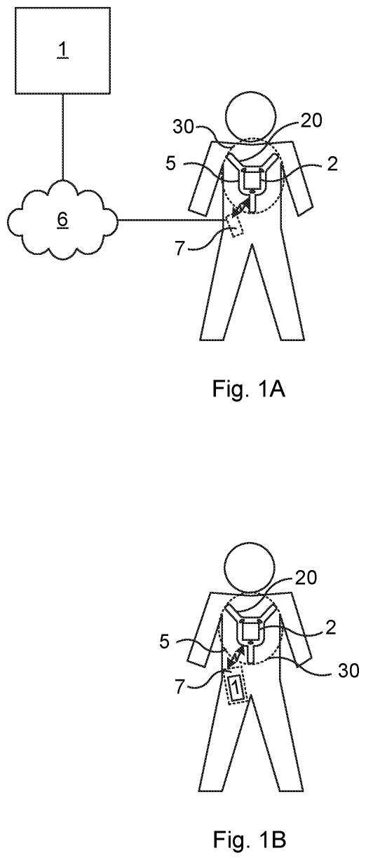

FIGS. 1A-B are schematic diagrams illustrating an environment in which embodiments presented herein can be applied.

Looking first to FIG. 1A, it is here shown a user 5 having a portable sensor device 2 provided in an electrode extension assembly 20. The electrode extension assembly 20 is fastened to the user 5, more specifically to the chest of the user 5. The electrode extension assembly 20 comprises electrodes in conductive contact with the user 5. The electrodes of the electrode extension assembly 20 are conductively connected to corresponding electrodes of the portable sensor device 2.

The user 5 also carries a smartphone 7 e.g. in a pocket. The portable sensor device 2 and the smartphone 7 can communicate over any suitable wireless interface, e.g. using Bluetooth or Bluetooth Low Energy (BLE), ZigBee, any of the IEEE 802.11x standards (also known as WiFi), etc.

The smartphone 7 is also connected to a wide area network 6, such as the Internet, e.g. via WiFi or a cellular network, to allow communication with an analysis device 1, here in the form of a server. The portable sensor device 2 captures ECG data and PCG data, via the electrode extension assembly 20 and sends this data, via the smartphone 7, to the analysis device 1. This allows the analysis device 1 to determine whether the heart of the user 5 can be considered to be in a normal state or whether the heart needs further examination based on the PCG data and the ECG data captured by the portable sensor device 2. Further investigation can be determined to be needed e.g. if any abnormal heart condition cannot be ruled out. It is to be noted that even if further investigation is to be performed, the heart can in fact be normal, i.e. non-pathological.

Together, the electrode extension assembly 20 and the portable sensor device 2 is denoted a sensor kit 30.

In FIG. 1B, the smartphone 7 contains the analysis device 1. In this way, the analysis can be performed locally, without the need for immediate access to the wide area network.

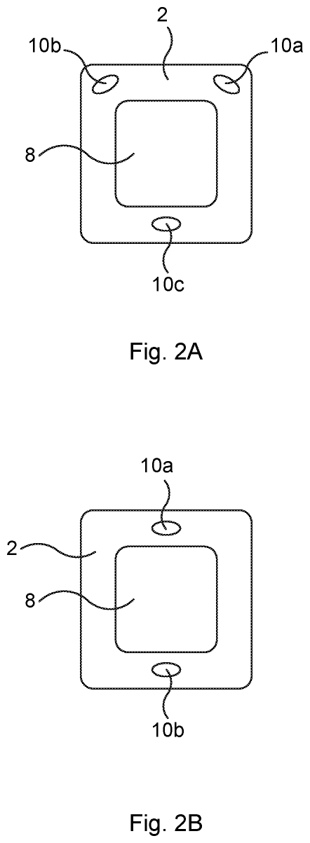

FIGS. 2A-2B are schematic diagrams of views illustrating a physical representation of the portable sensor device 2 of FIG. 1 according to one embodiment.

In FIG. 2A, a bottom view of the portable sensor device 2 is shown. There are here a first electrode boa, a second electrode bob and a third electrode loc. In order to capture the ECG data, the electrodes boa-c need to make electrical contact with the body of the user. This occurs when the portable sensor device 2 is placed in the electrode extension assembly, making conductive contact with the electrode extension assembly, which in turn makes conductive contact with the skin of the user. Using the electrodes, one or more analogue ECG signals are captured. The analogue ECG signals are converted to digital ECG signals using an analogue to digital (A/D) converter. The digital ECG signal is then sent to the analysis device for analysis together with the PCG signal.

Additionally, an audio sensor 8, e.g. in the form of a microphone, is provided to convert sound waves originating from the body into electric analogue PCG signals. The analogue PCG signals are converted to digital PCG signals using an A/D converter. The digital PCG signal is then sent to the analysis device for analysis together with the ECG signal.

It is to be noted that the portable sensor device 2 may also optionally comprise one or more user interface elements (not shown), e.g. push buttons, Light Emitting Diodes (LEDs), a display, a speaker, a user microphone, etc.

In FIG. 2B, a bottom view of a portable sensor device 2 similar to the one shown in FIG. 2A is shown. Here, however, the portable sensor device 2 comprises two electrodes boa-b.

It is to be noted that the portable sensor device 2 could also be provided with four electrodes or any other suitable number of electrodes.

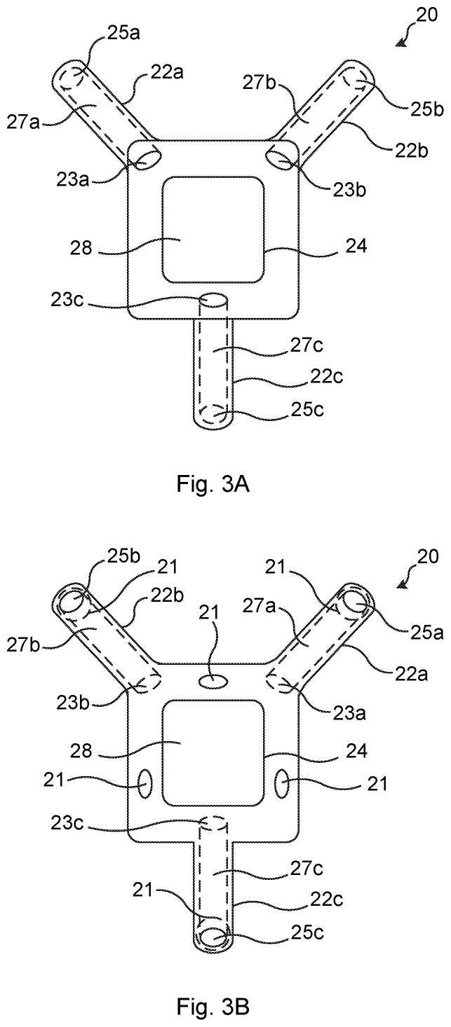

FIGS. 3A-B are schematic diagrams illustrating the electrode extension assembly 20 of FIG. 1 according to one embodiment.

A device holder 24 is provided for releasably holding a portable sensor device 2. As explained above, the portable sensor device 2 is configured to capture ECG signals and audio (PCG) signals. In one embodiment, the device holder 24 comprises one or more snap fastener to keep the portable sensor device 2 in place. In any case, the device holder 24 is adapted to receive a portable sensor device 2.

There are at least two extenders extending radially outwards from the device holder 24. The number of extenders can correspond to the number of electrodes of the portable sensor device 2. In this example, there are three extenders 22a-c corresponding to three electrodes of the portable sensor device 2.

Each extender 22a-c comprises a connector 23a-c configured to connect to a respective electrode 10a-c (see FIG. 2A) of the portable sensor device 2, when located in the device holder 24.

Moreover, each extender 22a-c comprises an outer electrode 25a-c connected at a distal end of the extender 22a-c. The outer electrode 25a-c is configured to be applied to a user 5, when the electrode extension assembly 20 is fastened to a user 5. Specifically, the outer electrode 25a-c is configured to make conductive contact with the skin of the user, e.g. on the chest. In this top view, the outer electrodes 25a-c are obscured by other parts of the extenders 22a-c, since the outer electrodes 25a-c are provided on the bottom of the extenders 22a-c.

Each extender 22a-c further comprises a conductor 27a-c connected on one end to the connector 23a-c and on the other end to the outer electrode 25a-c. This provides a conductive path from the skin of the user, via the outer electrodes 25a-c, the conductors 27a-c, the connectors 23a-c to the electrodes boa-c of the portable sensor device 2.

An audio signal conveyor 28 is provided for conveying audio (i.e. PCG) signals, when the electrode assembly 20 is fastened to a user and a portable sensor device 2 is located in the device holder 24. The audio signal conveyor 28 thus conveys audio from the user to an audio sensor 8 of the portable sensor device 2.

The audio signal conveyor 28 can be a through hole provided (when the electrode extension assembly is in use) between the user and the audio sensor of the portable sensor device 2. Optionally, the through hole can be filled with audio conductive material, such as foam or similar.

The electrode extension assembly optionally comprises conduits between the body and additional sensors of the portable sensor device. For instance, such additional sensors can include any one or more of additional microphones, temperature sensors etc. That are in acoustic or electrical conductive contact with the portable sensor device.

In FIG. 3B, a bottom view of the electrode extension assembly 20 is shown. Here, the fasteners 21 of the electrode extension assembly 20 can be seen. The electrode extension assembly comprises one or more fasteners 21 for releasably fastening the electrode extension assembly 20 to a user 5. The fasteners can e.g. comprises an adhesive for fastening the electrode extension assembly 20 to a user. The adhesive can e.g. be an acrylic, medical grade, adhesive. In this example, there are six fasteners 21, three on the respective extenders 22a-c and three on the central part of the electrode extension assembly 20. However, any suitable number of fasteners of any suitable shape can be provided, as long as the fasteners keep the electrode extension assembly 20 and inserted portable sensor device 2 securely attached to the body.

The electrode extension assembly 20 can be disposable. For instance, the electrodes, connectors and conductors can be made from conductive carbon, while the rest of the electrode extension assembly 20 can be made from plastic. This makes production simple and cheap, whereby the user can use a new electrode extension assembly 20 for each time PCG & ESG measurements are to be captured, and dispose of the electrode extension assembly 20 when it has been used only once. The portable sensor device 2 is of course reused between instances of measurement.

The electrode extension assembly 20 and the portable sensor device 2 of FIG. 1 are thus used together to used to capture measurements for ECG and for PCG. In order to capture measurements for ECG and for PCG, the portable sensor device 2 is placed inside the electrode extension assembly 20. The electrode extension assembly 20 is fastened on the skin body of the user, e.g. on the chest of the user, close to the heart of the user. The user can then cover the electrode extension assembly 20 and the portable sensor device 2, e.g. by wearing covering clothes.

In this way, the user can perform any activity while ECG and PCG measurements are captured. For instance, measurements can be captured in the daily life of the user, giving much more valuable data and covering a longer time period than if the user has to actively measure each time.

Using the electrode extension assembly 20, a distance between electrodes on the user is increased, which improves measurements of ECG. Moreover, by fastening the electrode extension assembly 20 including a portable sensor device 2 on the body, the user does not need to hold the portable sensor device 2 during measurements. This is beneficial, since when the user holds the portable sensor device 2 manually during measurement, small movements cause disturbances, particularly in the PCG data. Consequently, fastening the electrode extension assembly 20 improves data quality. The presented embodiments further enable measurement of body electrical impedance for recording of cardiac function or respiration through fixed positioning of ECG electrodes.

The invention has mainly been described above with reference to a few embodiments. However, as is readily appreciated by a person skilled in the art, other embodiments than the ones disclosed above are equally possible within the scope of the invention, as defined by the appended patent claims.

What is claimed is:

1. An electrode extension assembly comprising:
a fastener for releasably fastening the electrode extension assembly to a user;
a device holder for releasably holding a portable sensor device-configured to capture electrocardiogram signals and audio signals;
at least two extenders extending radially outwards from the device holder, wherein each extender comprises:
a connector configured to connect to a respective electrode of the portable sensor device, when located in the device holder;
an outer electrode connected at a distal end of the extender, the outer electrode being configured to be applied to a user, when the electrode extension assembly is fastened to a user;
a conductor connected on one end to the connector and on the other end to the outer electrode; and
an audio signal conveyors for conveying audio signals, when the electrode assembly is fastened to a user and a portable sensor device is located in the device holder, from the user to an audio sensor of the portable sensor device.

2. The electrode extension assembly according to claim 1, wherein the fastener comprises an adhesive for fastening the electrode extension assembly to a user.

3. The electrode extension assembly according to claim 1, comprising three extenders.

4. The electrode extension assembly according to claim 1, wherein the audio signal conveyor is a through hole provided, when the electrode assembly is fastened to a user and a portable sensor device is located in the device holder, between the user and the audio sensor of the portable sensor device.

5. The electrode extension assembly according to claim 1, wherein the device holder comprises a snap fastener.

6. The electrode extension assembly according to claim 1, wherein the electrode extension assembly is disposable.

7. A sensor kit comprising the electrode extension assembly according to claim 1 and a portable sensor device to be located in the device holder of the electrode extension assembly.
